(19) United States
(12) Patent Application Publication
(10) Pub. No.: US 2025/0143644 A1
(43) Pub. Date: May 8, 2025

(54) SMART RING WITH A PHYSIOLOGICAL FEATURE DETECTION FUNCTION AND RELATED PHYSIOLOGICAL FEATURE DETECTING METHOD

(71) Applicant: PixArt Imaging Inc., Hsin-Chu City (TW)

(72) Inventors: Yung-Chang Lin, Hsin-Chu City (TW); Jian-Cheng Liao, Hsin-Chu City (TW); Chun-Chih Chen, Hsin-Chu City (TW)

(73) Assignee: PixArt Imaging Inc., Hsin-Chu City (TW)

(21) Appl. No.: 19/015,669

(22) Filed: Jan. 10, 2025

Related U.S. Application Data
(63) Continuation-in-part of application No. 18/411,033, filed on Jan. 12, 2024.
Application No. 18/411,033 is a continuation-in-part of application No. 17/835,930, filed on Jun. 8, 2022.
Application No. 17/835,930 is a continuation-in-part of application No. 18/118,152, filed on Mar. 7, 2023.
(60) Provisional application No. 63/357,009, filed on Jun. 30, 2022.

(30) Foreign Application Priority Data
Jun. 11, 2021 (CN) 202110656442.1
May 5, 2022 (CN) 202210481440.8
Dec. 27, 2022 (CN) 202211680498.1

(51) Int. Cl. A61B 5/00

(52) U.S. Cl. CPC A61B 5/6843; A61B 5/6826; A61B 2560/0223; A61B 2560/0462; A61B 2562/0219; A61B 2562/0247; A61B 2562/043

(57) ABSTRACT

A smart ring with a physiological feature detecting method is applied to a target object. The smart ring includes a pressure sensor and an operation processor. The pressure sensor is adapted to detect a pressure value of the target object applied for the smart ring. The operation processor is electrically connected with the pressure sensor, and adapted to compare the pressure value with a preset condition and determine a behavior of the target object according to a comparison result of the pressure value and the preset condition for generating a related operation command.

### CROSS REFERENCE TO RELATED APPLICATIONS

This application is a continuation-in-part of U.S. application Ser. No. 18/411,033, filed on Jan. 12, 2024, which is a continuation-in-part of U.S. application Ser. No. 17/835,930, filed on Jun. 8, 2022, and is a continuation-in-part of U.S. application Ser. No. 18/118,152, filed on Mar. 7, 2023, which claims the benefit of U.S. Provisional Application No. 63/357,009, filed on Jun. 30, 2022. The contents of these applications are incorporated herein by reference.

### BACKGROUND OF THE INVENTION

### 1. Field of the Invention

The present invention relates to a smart ring and more particularly, to a smart ring with a physiological feature detection function and a related physiological feature detecting method.

### 2. Description of the Prior Art

A conventional smart ring utilizes the photoplethysmography (PPG) sensor to detect physiological features of the user, such as the heart rate or the blood oxygen level; however, the detection result of the physiological features are easily affected by the wearing posture of the smart ring to reduce the detection accuracy of the PPG sensor. For example, if the user wears the smart ring on the wrong finger, the detection accuracy of the PPG sensor is affected by tightness of the finger where on the smart ring is worn; even when the user wears the smart ring on the correct finger, the detection accuracy of the PPG sensor is still affected by palm gesture as if the detection area of the smart ring is excessive pressed. In addition, the convention smart ring only detects the physiological features of the user and has no additional functions. Design of a smart ring that can detect the correctness of wearing gesture to improve the detection accuracy and have other novel functions is an important issue in the smart portable device industry.

### SUMMARY OF THE INVENTION

The present invention provides a smart ring with a physiological feature detection function and a related physiological feature detecting method for solving above drawbacks.

According to the claimed invention, a smart ring with a physiological feature detection function is applied to a target object. The smart ring includes a pressure sensor and an operation processor. The pressure sensor is adapted to detect a pressure value of the target object applied for the smart ring. The operation processor is electrically connected with the pressure sensor, and adapted to compare the pressure value with a preset condition and determine a behavior of the target object according to a comparison result of the pressure value and the preset condition for generating a related operation command.

According to the claimed invention, the pressure sensor is set as a photoplethysmography sensor adapted to acquire a physiological detection value of the target object, and the operation processor is adapted to analyze variation of the physiological detection value to convert into the pressure value.

According to the claimed invention, the smart ring further includes a photoplethysmography sensor separated from the pressure sensor and electrically connected with the operation processor, and adapted to acquire a physiological detection value of the target object. The operation processor utilizes a sensing element of the pressure sensor to directly acquire and analyze the pressure value, and calibrate the physiological detection value in accordance with an analysis result of the pressure value.

According to the claimed invention, the operation processor decides the behavior of the target object to generate the related operation command when the physiological detection value conforms to another preset condition and the pressure value exceeds a threshold of the preset condition. The smart ring further includes a ring body, and the pressure sensor and the photoplethysmography sensor are disposed on adjacent positions inside the ring body.

According to the claimed invention, the smart ring further includes a ring body and a detection light source, the detection light source and the pressure sensor are disposed inside the ring body. The detection light source is at least one of a transmissive light source and a reflective light source. The detection light source includes the transmissive light source and the reflective light source, the operation processor alternately activates the transmissive light source and the reflective light source.

According to the claimed invention, the smart ring further includes an acceleration detector electrically connected with the operation processor and adapted to detect an acceleration value of the smart ring in each axis. The smart ring further includes a ring body and two pressure sensors, and the pressure sensors are respectively disposed on two opposite positions inside the ring body.

According to the claimed invention, a physiological feature detecting method is applied to a smart ring having a pressure sensor and an operation processor. The physiological feature detecting method includes acquiring a pressure value of the target object applied for the smart ring detected by the pressure sensor, comparing the pressure value with a preset condition, and determining a behavior of the target object according to a comparison result of the pressure value and the preset condition for generating a related operation command.

The pressure sensor of the smart ring can be the PPG sensor; the physiological detection value acquired by the PPG sensor can have obvious change when the smart ring is pressed by the target object, and the smart ring can analyze the variation of the physiological detection value to convert into the pressure value. The pressure sensor of the smart ring can further be separated from the PPG sensor, and use the sensing element such as the piezoelectric material or the metal membrane to directly acquire the pressure value, so that the smart ring can calibrate the physiological detection value of the PPG sensor by the analysis result of the pressure value. The smart ring of the present invention can utilize the pressure sensor to calibrate the detection result of the PPG sensor, and further utilize the pressure sensor to detect tightness, gesture, or finger motion of the user when wearing the smart ring.

These and other objectives of the present invention will no doubt become obvious to those of ordinary skill in the art after reading the following detailed description of the preferred embodiment that is illustrated in the various figures and drawings.

### BRIEF DESCRIPTION OF THE DRAWINGS

FIG. 1 is an application diagram of a smart ring according to an embodiment of the present invention.

FIG. 2 is an appearance diagram of the smart ring according to the embodiment of the present invention.

FIG. 3 is a sectional view of the smart ring according to the embodiment of the present invention.

FIG. 4 is a functional block diagram of the smart ring according to the embodiment of the present invention.

FIG. 5 is a flow chart of a physiological feature detecting method according to the embodiment of the present invention.

### DETAILED DESCRIPTION

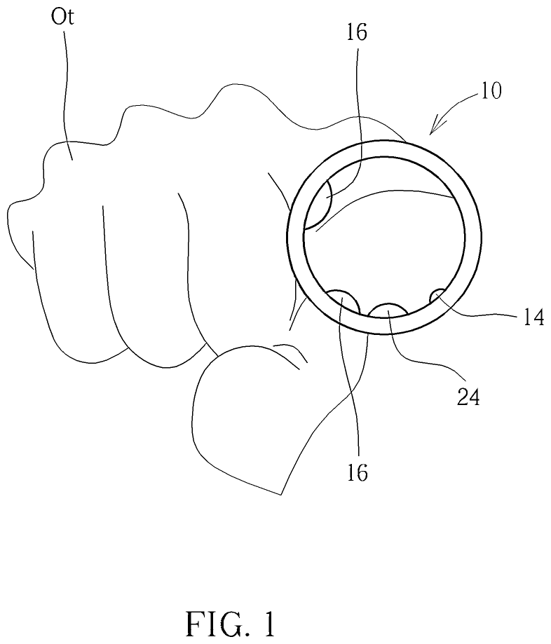

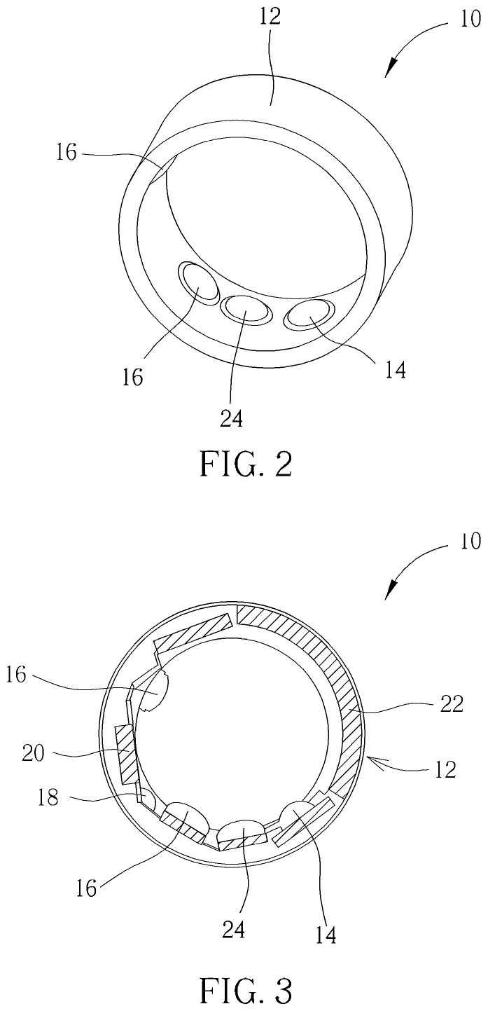

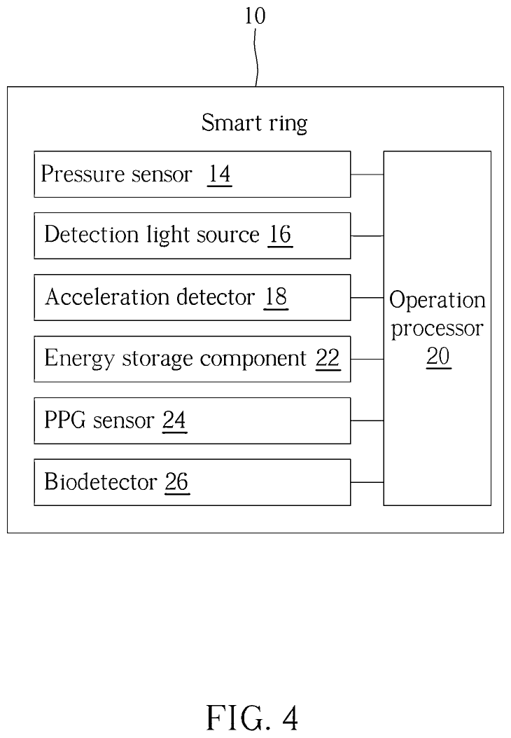

Please refer to FIG. 1 to FIG. 4. FIG. 1 is an application diagram of a smart ring 10 according to an embodiment of the present invention. FIG. 2 is an appearance diagram of the smart ring 10 according to the embodiment of the present invention. FIG. 3 is a sectional view of the smart ring 10 according to the embodiment of the present invention. FIG. 4 is a functional block diagram of the smart ring 10 according to the embodiment of the present invention. The smart ring 10 can be applied to a target object Ot, such as wearing on the finger, for providing a physiological feature detection function. Besides, the smart ring 10 of the present invention can provide a pressure detection function, a pressure value generated by the pressure detection function can calibrate a result of the physiological feature detection function for preferred detection accuracy, and can further generate an operation command in accordance with variation of the pressure value, so that the smart ring 10 can have more diverse applications.

The smart ring 10 can optionally include a ring body 12, a pressure sensor 14, a detection light source 16, an acceleration detector 18, an operation processor 20 and an energy storage component 22. The pressure sensor 14, the detection light source 16, the acceleration detector 18 and the energy storage component 22 can be electrically connected with the operation processor 20 and disposed inside the ring body 12. A shape of the ring body 12 can depend on a design demand. Generally, an inner annular surface of the ring body 12 can be designed as a round form to match with the finger, and an outer annular surface of the ring body 12 can be designed as the round form or a square form; possible variation of the ring body 12 is omitted herein for simplicity. The pressure sensor 14 can detect the pressure value of the target object Ot applied for the smart ring 10. For example, if the smart ring 10 is worn on the forefinger, the pressure sensor 14 can detect the pressure value of the forefinger (such as the target object Ot) applied for the inner annular surface of the smart ring 10, and further can detect the pressure value of the thumb or other fingers or other tools (such as the target object Ot) applied for the outer annular surface of the smart ring 10.

The detection light source 16 can be optionally designed as a transmissive light source and/or a reflective light source; if the detection light source 16 has both the transmissive light source and the reflective light source, the operation processor 20 can optionally activate the transmissive light source or the reflective light source, or can alternately activate the transmissive light source and the reflective light source according to the design demand (e.g., the transmissive light source consumes more energy but has high accuracy). The detection light source 16 can be a combination of green light units, a red light unit and an infrared light unit, or can be a combination of a yellow light unit, the green light unit and the infrared light unit; its variation depends on an actual demand. Generally, the reflective light source of the detection light source 16 can be positioned between the pressure sensor 14 and the operation processor 20, and the reflective light source of the detection light source 16 can be positioned between the operation processor 20 and the energy storage component 22, but the actual application is not limited thereto. Moreover, the acceleration detector 18 can detect an acceleration value of the smart ring 10 in each axis, which helps to accurately determine changes in the user's gestures during walking or running when wearing the smart ring 10. The energy storage component 22 can include a charging unit and a storage unit; the charging unit can be a wireless charger used to transmit external energy into the storage unit and then transfer to other electronic components of the smart ring 10.

In one embodiment of the present invention, the pressure sensor 14 can be defined as a photoplethysmography sensor used to acquire a physiological detection value of the target object Ot. The operation processor 20 can analyze the physiological detection value generated by the photoplethysmography sensor to determine pressure. For example, the operation processor 20 can analyze whether the physiological detection value has abnormal change within a specific time period, and analyze difference between a normal value and the abnormal change of the physiological detection value to convert the foresaid difference into the pressure value; then, the operation processor 20 can compare the pressure value with a preset condition, and determine a behavior of the target object Ot in accordance with a comparison result of the pressure value and the preset condition, for generating a related operation command. The preset condition may be a threshold or a numeric range; situations of the pressure value higher than or lower than the threshold, or the pressure value higher than or lower than or within the numeric range can be indicated as different behaviors of the target object Ot and different operation commands.

In another embodiment of the present invention, the smart ring 10 can include a photoplethysmography sensor (PPG sensor) 24 separated from the pressure sensor 14. The pressure sensor 14 and the PPG sensor 24 can respectively acquire the pressure value and the physiological detection value from the target object Ot. In this embodiment, the pressure sensor 14 and the PPG sensor 24 can be preferably disposed on adjacent positions inside the ring body 12. When the smart ring 10 is worn on the target object Ot, the pressure sensor 14 and the PPG sensor 24 can simultaneously contact the same or adjacent areas on the target object Ot, so as to ensure that the pressure value of the pressure sensor 14 and the physiological detection value of the PPG sensor 24 can have optimal homogeneity for complementary calibration.

In the embodiment of the pressure sensor 14 separated from the PPG sensor 24, the pressure sensor 14 can optionally utilize piezoelectric material, metal membrane, or any sensing element that conforms to the actual demand to directly acquire and analyze the pressure value. If the target object Ot applies excessive pressure on the smart ring 10, the physiological detection value of the target object Ot acquired by the PPG sensor 24 can have the abnormal change, and the pressure value applied by the target object Ot and acquired by the pressure sensor 14 may exceed the preset condition (such as the threshold or the numeric range mentioned as above). Therefore, the operation processor 20 can compare the pressure value with the preset condition; if the pressure value does not conform to the preset condition (for example, the pressure value may be lower than the threshold or the numeric range), the pressure value is within the normal range, and the user wears the smart ring 10 correctly, so that the smart ring 10 can transmit the physiological detection value of the PPG sensor 24 to the external device (such as the smart phone).

If the pressure value conforms to the preset condition (for example, the pressure value may be greater than or equal to the threshold or the numeric range), the pressure value is not within the normal range, and the user wears the smart ring 10 incorrectly, so that the smart ring 10 may delete the related physiological detection value, and output a computation value (such as a mean value) of physiological detection values acquired in a previous period when the pressure value is within the normal range to an external device; or the smart ring 10 can optionally utilize the computation value of the physiological detection value to calibrate the physiological detection value acquired in a current period. That is to say, the smart ring 10 can confirm whether the physiological detection value of the PPG sensor 24 conforms to another preset condition, for determining that the target object Ot is not in a stationary state when the physiological detection value is varied and conforms to another preset condition, and then deciding the behavior (for example, whether the smart ring 10 is worn correctly or has other pressure change) of the target object Ot to generate the related operation command when the pressure value conforms to (or exceeds) the preset condition. The foresaid another preset condition can be set by detection sensitivity of the smart ring 10; methods of the physiological detection value calibrated by the pressure value are not limited to the foresaid embodiment, and depend on the design demand.

In a possible embodiment of the present invention, the smart ring 10 can optionally include two pressure sensors 14. One pressure sensor 14 can be preferably disposed adjacent to the PPG sensor 24, and the other pressure sensor (which is not shown in the figures) can be disposed inside the ring body 12 and on position opposite to the PPG sensor 24 (or opposite to the adjacent pressure sensor 14). When the smart ring 10 is worn on the target object Ot (such as the finger), the operation processor 20 can decide the smart ring 10 is correctly worn on the finger if the pressure sensor 14 next to the PPG sensor 24 detects the pressure value within the normal range; if the pressure sensor 14 next to the PPG sensor 24 detects the pressure value within the abnormal range, the smart ring 10 can analyze the pressure value acquired by the pressure sensor (which is in the opposite position and not shown in the figures) to decide whether the smart ring 10 is worn on the finger incorrectly. For example, when the user's palm faces upwardly to press the pressure sensor (which is not shown in the figures) of the smart ring 10 that is near the back of the finger, the smart ring 10 can output a warning signal and accordingly calibrate the physiological detection value acquired by the PPG sensor 24 in this period.

Besides, the smart ring 10 can optionally dispose a biodetector 26 inside the ring body 12. The biodetector 26 can be a capacitive detector and/or an electrode detector used to detect whether the smart ring 10 is put on the organism. For example, if the smart ring 10 is placed on the bright table, the smart ring 10 may misjudge to have been worn on the finger because a detection signal of the PPG sensor is reflected by the bright table. The smart ring 10 can have the biodetector 26, and the smart ring 10 can decide that the detection signal of the PPG sensor is interpreted as the physiological detection value when the detection signals of the PPG sensor and the biodetector 26 conform to related conditions.

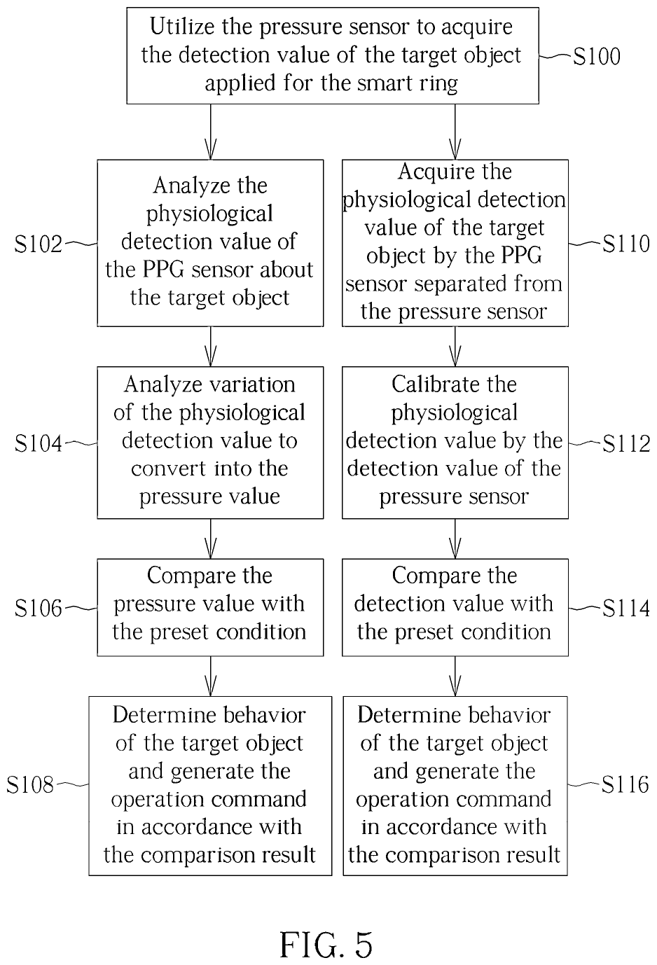

Please refer to FIG. 5. FIG. 5 is a flow chart of a physiological feature detecting method according to the embodiment of the present invention. The physiological feature detecting method illustrated in FIG. 5 can be suitable for the smart ring 10 shown in FIG. 1 to FIG. 4. First, step S100 can be executed that the operation processor 20 can utilize the pressure sensor 14 to acquire the detection value of the target object Ot applied for the smart ring 10. If the smart ring 10 uses the PPG sensor as the pressure sensor 14, step S102 and step S104 can be executed to analyze the physiological detection value of the PPG sensor about the target object Ot, and analyze the variation of the physiological detection value to convert into the pressure value. Then, step S106 and step S108 can be executed to compare the pressure value with the preset condition, and determine the behavior of the target object Ot and generate the related operation command in accordance with the comparison result of the pressure value and the preset condition.

If the smart ring 10 uses the piezoelectric material, the metal membrane, or the sensing element with similar functions applied to the pressure sensor 14, step S110 and step S112 can be executed to acquire the physiological detection value of the target object Ot by the PPG sensor 24 separated from the pressure sensor 14, and calibrate the physiological detection value by the pressure value of the pressure sensor 14; the foresaid calibration method can be the same as content related to FIG. 1 to FIG. 4, and a detailed description is omitted herein for simplicity. Moreover, step S114 and step S116 can be executed to compare the detection value (which means the pressure value) with the preset condition, and determine the behavior of the target object Ot and generate the related operation command in accordance with the comparison result of the pressure value and the preset condition.

In process of step S108 and step S116, the smart ring 10 can detect whether the pressure value is abnormal for deciding the smart ring 10 is worn on the target object Ot correctly or incorrectly; the smart ring 10 can further detect the abnormal change of the pressure value in a specific period (such as a short period of time), and therefore determine whether the target object Ot where on the smart ring 10 is worn is bent or whether the smart ring 10 is hit by an external force. That is, the user can wear the smart ring 10, and the smart ring 10 can output the warning signal when the smart ring 10 is worn on the incorrect finger, or worn on the correct finger but the hand is in improper posture; when the smart ring 10 is worn on the forefinger correctly, the forefinger can be bent or the thumb can tap the smart ring 10 worn on the forefinger, and the smart ring 10 can detect the variation of the pressure value to generate the operation command, so that the smart ring 10 can replace the wheel and buttons of the mouse and have functions of sliding pages and clicking in the air. If the smart ring 10 has two pressure sensors 14, the smart ring 10 can provide left and right click functions of the mouse.

In conclusion, the pressure sensor of the smart ring can be the PPG sensor; the physiological detection value acquired by the PPG sensor can have obvious change when the smart ring is pressed by the target object, and the smart ring can analyze the variation of the physiological detection value to convert into the pressure value. The pressure sensor of the smart ring can further be separated from the PPG sensor, and use the sensing element such as the piezoelectric material or the metal membrane to directly acquire the pressure value, so that the smart ring can calibrate the physiological detection value of the PPG sensor by the analysis result of the pressure value. The pressure sensor of the smart ring can be attached to the skin of the finger to detect changes in pressure during various finger movements, such as bending the finger, tapping on a table, or pinching fingers together. The smart ring of the present invention can utilize the pressure sensor to calibrate the detection result of the PPG sensor, and further utilize the pressure sensor to detect tightness, gesture, or finger motion of the user when wearing the smart ring.

Those skilled in the art will readily observe that numerous modifications and alterations of the device and method may be made while retaining the teachings of the invention. Accordingly, the above disclosure should be construed as limited only by the metes and bounds of the appended claims.

What is claimed is:

1. A smart ring with a physiological feature detection function and applied to a target object, the smart ring comprising:
a pressure sensor adapted to detect a pressure value of the target object applied for the smart ring; and
an operation processor electrically connected with the pressure sensor, and adapted to compare the pressure value with a preset condition and determine a behavior of the target object according to a comparison result of the pressure value and the preset condition for generating a related operation command.

2. The smart ring of claim 1, wherein the pressure sensor is set as a photoplethysmography sensor adapted to acquire a physiological detection value of the target object, the operation processor is adapted to analyze variation of the physiological detection value to convert into the pressure value.

3. The smart ring of claim 1, wherein the smart ring further comprises a photoplethysmography sensor separated from the pressure sensor and electrically connected with the operation processor, and adapted to acquire a physiological detection value of the target object.

4. The smart ring of claim 3, wherein the operation processor utilizes a sensing element of the pressure sensor to directly acquire and analyze the pressure value, and calibrate the physiological detection value in accordance with an analysis result of the pressure value.

5. The smart ring of claim 3, wherein the operation processor decides the behavior of the target object to generate the related operation command when the physiological detection value conforms to another preset condition and the pressure value exceeds a threshold of the preset condition.

6. The smart ring of claim 3, wherein the smart ring further comprises a ring body, the pressure sensor and the photoplethysmography sensor are disposed on adjacent positions inside the ring body.

7. The smart ring of claim 1, wherein the smart ring further comprises a ring body and a detection light source, the detection light source and the pressure sensor are disposed inside the ring body.

8. The smart ring of claim 7, wherein the detection light source is at least one of a transmissive light source and a reflective light source.

9. The smart ring of claim 8, wherein the detection light source comprises the transmissive light source and the reflective light source, the operation processor alternately activates the transmissive light source and the reflective light source.

10. The smart ring of claim 1, wherein the smart ring further comprises an acceleration detector electrically connected with the operation processor and adapted to detect an acceleration value of the smart ring in each axis.

11. The smart ring of claim 1, wherein the smart ring further comprises a ring body and two pressure sensors, the pressure sensors are respectively disposed on two opposite positions inside the ring body.

12. A physiological feature detecting method applied to a smart ring having a pressure sensor and an operation processor, the physiological feature detecting method comprising:
the operation processor acquiring a pressure value of the target object applied for the smart ring detected by the pressure sensor;
the operation processor comparing the pressure value with a preset condition; and
the operation processor determining a behavior of the target object according to a comparison result of the pressure value and the preset condition for generating a related operation command.

13. The physiological feature detecting method of claim 12, wherein the pressure sensor is set as a photoplethysmography sensor, the physiological feature detecting method further comprises:
the operation processor determining whether a physiological detection value of the target object acquired by the photoplethysmography sensor is varied; and
the operation processor analyzing variation of the physiological detection value to convert into the pressure value.

14. The physiological feature detecting method of claim 12, wherein the smart ring comprises a photoplethysmography sensor separated from the pressure sensor, the physiological feature detecting method further comprises:
the operation processor utilizing the photoplethysmography sensor to acquire a physiological detection value of the target object;
the operation processor utilizing a sensing element of the pressure sensor to directly acquire and analyze the pressure value; and
the operation processor calibrating the physiological detection value in accordance with an analysis result of the pressure value.

15. The physiological feature detecting method of claim 14, wherein the physiological feature detecting method further comprises:
the operation processor deciding the behavior of the target object to generate the related operation command when the physiological detection value conforms to another preset condition and the pressure value exceeds a threshold of the preset condition.

16. The physiological feature detecting method of claim 12, wherein the smart ring comprises a detection light source having a transmissive light source and a reflective light source, the physiological feature detecting method further comprises:
the operation processor alternately activating the transmissive light source and the reflective light source in operation process of the pressure sensor.

17. The physiological feature detecting method of claim 12, wherein the smart ring comprises two pressure sensors respectively disposed on opposite positions, the physiological feature detecting method further comprises:
the operation processor utilizing pressure values detected by the two pressure sensors to determine whether the smart ring is correctly worn on the target object.
